(19) United States
(12) Patent Application Publication
(10) Pub. No.: US 2017/0217962 A1
(43) Pub. Date: Aug. 3, 2017

(54) A PROCESS FOR THE PREPARATION OF PALBOCICLIB

(71) Applicant: Sun Pharmaceutical Industries Limited, Mumbai, Maharashtra (IN)

(72) Inventors: Vipin TYAGI, Gurgaon (IN); Kallimulla MOHAMMAD, Krishna (IN); Bishwa Prakash RAI, Azamgarh (IN); Mohan PRASAD, Gurgaon (IN)

(21) Appl. No.: 15/500,616

(22) PCT Filed: Jul. 21, 2015
(86) PCT No.: PCT/IB15/55528

§ 371 Date: Jan. 31, 2017

(30) Foreign Application Priority Data
Jul. 31, 2014 (IN) 2154/DEL/2014

(51) Int. Cl. C07D 471/04; C07F 7/18

(52) U.S. Cl. CPC C07D 471/04; C07F 7/1892

(57) ABSTRACT

The present invention relates to a process for the preparation of palbociclib utilizing a silyl-protected crotonic acid derivative to produce a silyl-protected 2-chloro-4-(cyclopentylamino)-5-(1-methyl-2-carboxy-ethen-1-yl)pyrmidine followed by intramolecular cyclization of the pyrmidine intermediate to produce 2-chloro-8-cyclopentyl-5-methyl-8H-pyrido[2,3-d]pyrimidin-7-one which is then converted to palbociclib.

### FIELD OF THE INVENTION

The present invention relates to a process for the preparation of palbociclib.

### BACKGROUND OF THE INVENTION

Palbociclib chemically is 6-acetyl-8-cyclopentyl-5-methyl-2-[[5-(1-piperazinyl)-2-pyridinyl]amino]pyrido[2,3-d]pyrimidin-7(8H)-one, represented by the Formula I.

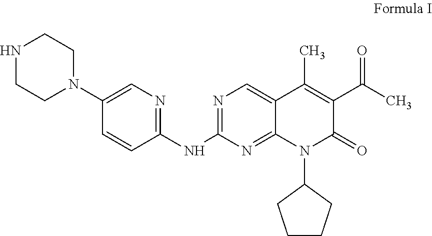 [SMILES: CC(=O)c1c(C)c2cnc(Nc3ccc(N4CCNCC4)cn3)nc2n(C2CCCC2)c1=O]

U.S. Pat. No. 6,936,612 discloses palbociclib and a process for the preparation of its hydrochloride salt.

U.S. Pat. No. 7,781,583 discloses a process for the preparation of palbociclib, wherein 2-chloro-8-cyclopentyl-5-methyl-8H-pyrido[2,3-d]pyrimidin-7-one of Formula II

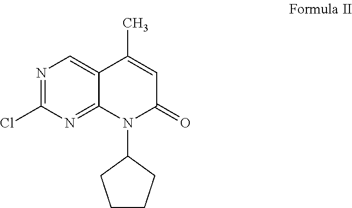 [SMILES: Cc1cc(=O)n(C2CCCC2)c2nc(Cl)ncc12]

is prepared by reacting 5-bromo-2-chloro-N-cyclopentylpyrimidin-4-amine of Formula III

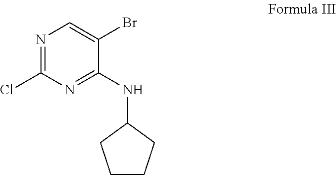 [SMILES: Clc1ncc(Br)c(NC2CCCC2)n1]

with crotonic acid.

U.S. Pat. No. 7,863,278 discloses polymorphs of various salts of palbociclib and processes for their preparation.

### SUMMARY OF THE INVENTION

The present invention relates to a process for the preparation of palbociclib.

### DETAILED DESCRIPTION OF THE INVENTION

The term “about,” as used herein, refers to any value which lies within the range defined by a number up to ±10% of the value.

The term “room temperature,” as used herein, refers to a temperature in the range of 25° C. to 35° C.

A first aspect of the present invention provides a process for the preparation of a compound of Formula IV,

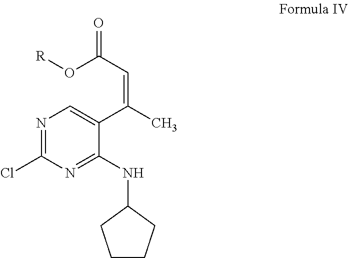

wherein R is trimethylsilyl, dimethylsilyl, or tert-butyldimethylsilyl
comprising reacting a crotonic acid derivative of Formula V

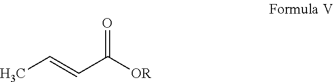

wherein R is trimethylsilyl, dimethylsilyl, or tert-butyldimethylsilyl
with a compound of Formula III

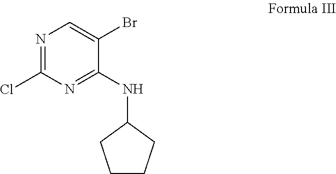 [SMILES: Clc1ncc(Br)c(NC2CCCC2)n1]

in the presence of a palladium catalyst, a base, and optionally a ligand to give a compound of Formula IV.

A second aspect of the present invention provides a process for the preparation of palbociclib of Formula I,

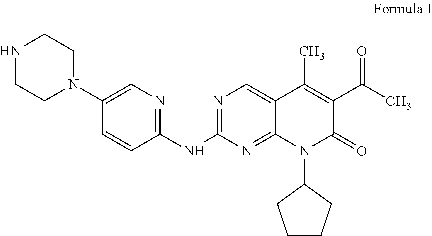 [SMILES: CC(=O)c1c(C)c2cnc(Nc3ccc(N4CCNCC4)cn3)nc2n(C2CCCC2)c1=O]

comprising:

a) reacting a crotonic acid derivative of Formula V,

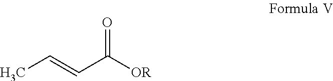

wherein R is trimethylsilyl, dimethylsilyl, or tert-butyldimethylsilyl
with a compound of Formula III

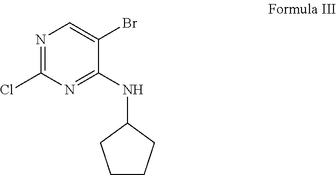 [SMILES: Clc1ncc(Br)c(NC2CCCC2)n1]

in the presence of a palladium catalyst, a base, and optionally a ligand to give a compound of Formula IV

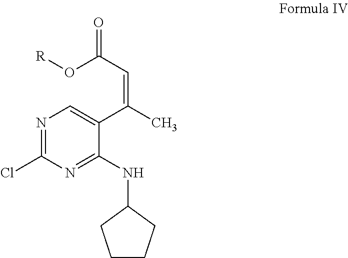

wherein R is trimethylsilyl, dimethylsilyl, or tert-butyldimethylsilyl; and

b) converting the compound of Formula IV to palbociclib of Formula I.

A third aspect of the present invention provides a process for the preparation of a compound of Formula II

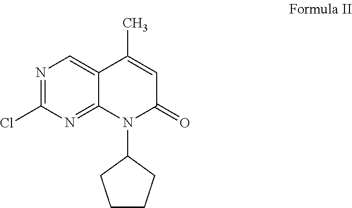 [SMILES: Cc1cc(=O)n(C2CCCC2)c2nc(Cl)ncc12]

comprising:
a) reacting a crotonic acid derivative of Formula V,

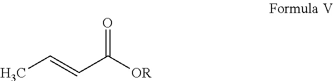

wherein R is trimethylsilyl, dimethylsilyl, or tert-butyldimethylsilyl
with a compound of Formula III

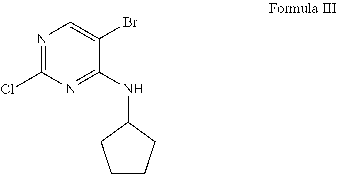 [SMILES: Clc1ncc(Br)c(NC2CCCC2)n1]

in the presence of a palladium catalyst, a base, and optionally a ligand to give a compound of Formula IV,

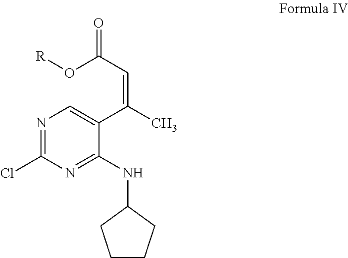

wherein R is trimethylsilyl, dimethylsilyl, or tert-butyldimethylsilyl; and
b) intramolecular cyclization of the compound of Formula IV to give a compound of Formula II.

A fourth aspect of the present invention provides a process for the preparation of palbociclib of Formula I

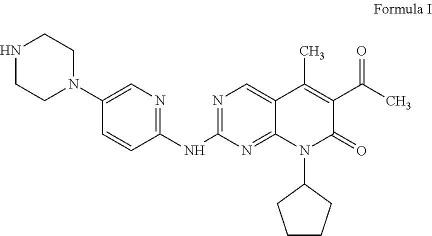 [SMILES: CC(=O)c1c(C)c2cnc(Nc3ccc(N4CCNCC4)cn3)nc2n(C2CCCC2)c1=O]

comprising:
a) reacting a crotonic acid derivative of Formula V,

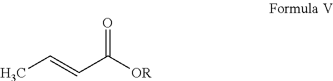

wherein R is trimethylsilyl, dimethylsilyl, or tert-butyldimethylsilyl
with a compound of Formula III

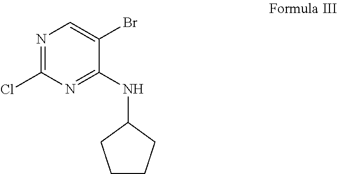 [SMILES: Clc1ncc(Br)c(NC2CCCC2)n1]

in the presence of a palladium catalyst, a base, and optionally a ligand to give a compound of Formula IV

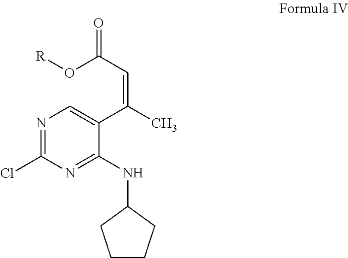

wherein R is trimethylsilyl, dimethylsilyl, or tert-butyldimethylsilyl;
b) intramolecular cyclization of the compound of Formula IV to give a compound of Formula II; and

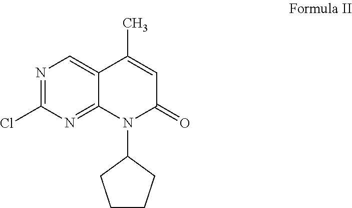 [SMILES: Cc1cc(=O)n(C2CCCC2)c2nc(Cl)ncc12]

c) converting the compound of Formula II to palbociclib of Formula I.

The compound of Formula V may be prepared by any method known in the art, for example, the method described in U.S. Pat. No. 7,126,025, or by the method as described herein.

The compound of Formula III may be prepared by any method known in the art, for example, the method described in U.S. Pat. No. 7,781,583.

The compound of Formula III is reacted with the compound of Formula V in the presence of the palladium catalyst, the base, and optionally the ligand to give the compound of Formula IV in a solvent.

The compound of Formula V may be reacted with the compound of Formula III after isolation from the reaction mixture in which it is formed. Alternatively, the reaction mixture containing the compound of Formula V may be used for the reaction with the compound of Formula III.

The base is an organic base or an inorganic base. Examples of organic bases include triethylamine, diisopropylethylamine, and tributylamine. Examples of inorganic bases include potassium carbonate, sodium carbonate, and lithium carbonate.

The palladium catalyst is selected from the group consisting of tetrakis(triphenylphosphine)palladium (0), palladium acetate, palladium chloride, and trans-dichlorobis(acetonitrile)palladium (II).

The ligand is selected from the group consisting of tri-o-tolylphosphine, triphenylphosphine, and tri-t-butylphosphine.

The solvent is selected from the group consisting of ethers, halogenated hydrocarbons, alcohols, and esters. Examples of ether solvents include tetrahydrofuran, 1,4-dioxane, diisopropylether, and methyl tert-butyl ether. Examples of halogenated hydrocarbon solvents include dichloromethane, dichloroethane, chloroform, and carbon tetrachloride. Examples of alcohol solvents include methanol, ethanol, n-propanol, isopropanol, and n-butanol. Examples of ester solvents include ethyl acetate and butyl acetate.

The reaction of the compound of Formula III with the compound of Formula V is carried out for from about 15 hours to about 30 hours, for example, from about 18 hours to about 24 hours.

The reaction of the compound of Formula III with the compound of Formula V is carried out at a temperature of from about 50° C. to about 90° C., for example, from about 70° C. to about 80° C.

The compound of Formula IV may optionally be isolated by filtration, decantation, extraction, distillation, evaporation, chromatography, precipitation, concentration, crystallization, centrifugation, or recrystallization. The compound of Formula IV may be dried using conventional techniques, for example, drying, drying under vacuum, spray drying, air drying, or agitated thin film drying.

The intramolecular cyclization of the compound of Formula IV to give the compound of Formula II is carried out in the presence of an acid anhydride or an acid chloride.

Examples of acid anhydrides include acetic anhydride, propionic anhydride, butyric anhydride, trifluoroacetic anhydride, and trifluoromethanesulfonic anhydride. Examples of acid chlorides include acetyl chloride and ethanoyl chloride.

The intramolecular cyclization of the compound of Formula IV may be carried out after isolation from the reaction mixture in which it is formed. Alternatively, the reaction mixture containing the compound of Formula IV may be used for this step.

The intramolecular cyclization of the compound of Formula IV is carried out for from about 1 hour to about 6 hours, for example, from about 2 hours to about 3 hours.

The intramolecular cyclization of the compound of Formula IV is carried out at a temperature of from about 50° C. to about 90° C., for example, from about 70° C. to about 80° C.

The compound of Formula II may optionally be isolated by filtration, decantation, extraction, distillation, evaporation, chromatography, precipitation, concentration, crystallization, centrifugation, or recrystallization. The compound of Formula II may be dried using conventional techniques, for example, drying, drying under vacuum, spray drying, air drying, or agitated thin film drying.

The compound of Formula II is converted to palbociclib of Formula I by processes known in the art, for example, as disclosed in U.S. Pat. No. 7,781,583.

While the present invention has been described in terms of its specific aspects and embodiments, certain modifications and equivalents will be apparent to those skilled in the art, and are intended to be included within the scope of the present invention.

### Method

Chromatographic purity was determined by HPLC using an Agilent® Model 1200; the column used was an ACE® C18-PFP (150×4.6 nm).

The following examples are for illustrative purposes only and should not be construed as limiting the scope of the invention in any way.

### Examples

### Preparation of 2-chloro-8-cyclopentyl-5-methyl-8H-pyrido[2,3-d]pyrimidin-7-one (Formula II)

### Step a: Preparation of trimethylsilyl (2E)-but-2-enoate (Formula V, when R is trimethylsilyl)

Crotonic acid (18.68 g) was taken in dichloromethane (80 mL) at room temperature to obtain a solution. Hexamethyldisilazane (HMDS) (21 g) followed by imidazole (0.4 g) was added to the solution at room temperature under stirring. The reaction mixture was refluxed for 2 hours. Dichloromethane was recovered completely under vacuum at 45° C. Dichloromethane (200 mL) was again added to the reaction mixture, and then recovered completely under vacuum at 45° C. The colorless liquid obtained was taken as such for next step.

### Step b: Preparation of 2-chloro-8-cyclopentyl-5-methyl-8H-pyrido[2,3-d]pyrimidin-7-one (Formula II)

### Method A

Trimethylsilyl (2E)-but-2-enoate (obtained from step a) and diisopropylethylamine (52 mL) were added to a solution of 5-bromo-2-chloro-N-cyclopentylpyrimidin-4-amine (20 g, Formula III) in tetrahydrofuran (100 mL) at room temperature under a nitrogen atmosphere. The reaction system was degassed under vacuum and then flushed with nitrogen; this evacuation procedure was repeated three times. Trans-dichlorobis(acetonitrile) palladium (II) (0.970 g) followed by the addition of tri-o-tolylphosphine (0.770 g) was added to the reaction mixture under a nitrogen atmosphere. The reaction system was again degassed under vacuum and then flushed with nitrogen; this evacuation procedure was repeated three times. The reaction mixture was heated at 75° C. to 80° C. overnight. The progress of the reaction was monitored by thin layer chromatography (TLC) (60% ethyl acetate/toluene). Trans-dichlorobis(acetonitrile) palladium (II) (0.725 g) was again added followed by the addition of tri-o-tolylphosphine (0.725 g) to the reaction mixture at 75° C. to 80° C. The reaction mixture was heated at 75° C. to 80° C. for 4 hours. After completion of the reaction, acetic anhydride (17 mL) was added, and then the mixture was stirred at 75° C. to 80° C. for 3 hours. The reaction mixture was cooled to room temperature. Dichloromethane (100 mL) and 1N hydrochloric acid (100 mL) were added and then the mixture was stirred for 10 minutes. The layers were separated and the aqueous layer was re-extracted with dichloromethane (40 mL) and separated. The combined organic layers were washed with a 5% sodium bicarbonate solution (200 mL) at room temperature. The organic layer was separated and activated carbon (2 g) was added to the mixture. The mixture was stirred for 20 minutes at room temperature. The mixture was filtered through a Hyflo® bed and then washed with dichloromethane (40 mL). The organic layer was evaporated under vacuum to obtain a residue. Isopropyl alcohol (80 mL) was added to the residue and the solvent was evaporated under reduced pressure until 40 mL of isopropyl alcohol remained. Isopropyl alcohol (40 mL) was again added to the mixture, and then the solvent was evaporated under reduced pressure until 20 mL of isopropyl alcohol remained. The mixture was stirred for 3 hours at room temperature. The product was filtered, then washed with isopropyl alcohol (20 mL), and then dried under vacuum at 45° C. to obtain the title compound.

Yield: 0.535% w/w

Chromatographic purity: 99.51%

### Method B

Trimethylsilyl (2E)-but-2-enoate (obtained from step a) and diisopropylethylamine (26.5 mL) were added to a solution of 5-bromo-2-chloro-N-cyclopentylpyrimidin-4-amine (Formula III, 10 g) in tetrahydrofuran (50 mL) at room temperature under a nitrogen atmosphere. The reaction system was degassed under vacuum and then flushed with nitrogen; this evacuation procedure was repeated three times. Trans-dichlorobis(acetonitrile) palladium (II) (1.39 g) followed by the addition of tri-o-tolylphosphine (1.1 g) was added to the reaction mixture under a nitrogen atmosphere. The reaction system was degassed under vacuum and then flushed with nitrogen; this evacuation procedure was repeated three times. The reaction mixture was heated at 75° C. to 80° C. overnight. After completion of the reaction, acetic anhydride (20 mL) was added, and then the mixture was stirred at 75° C. to 80° C. for 3 hours. The reaction mixture was cooled to room temperature. Dichloromethane (50 mL) and 1N hydrochloric acid (50 mL) were added, and then the mixture was stirred for 10 minutes. The layers were separated and the aqueous layer was re-extracted with dichloromethane (20 mL) and separated. The combined organic layers were washed with a 5% sodium bicarbonate solution (200 mL) at room temperature. The organic layer was separated and activated carbon (1 g) was added to the mixture. The mixture was stirred for 20 minutes at room temperature. The mixture was filtered through a Hyflo® bed and then washed with dichloromethane (20 mL). The organic layer was evaporated under vacuum to obtain a residue. Isopropyl alcohol (40 mL) was added to the residue and then the solvent was evaporated under reduced pressure until 20 mL of isopropyl alcohol remained. Isopropyl alcohol (20 mL) was again added to the mixture and then the solvent was evaporated under reduced pressure until 20 mL of isopropyl alcohol remained. The mixture was stirred for 3 hours at room temperature. The product was filtered and washed with isopropyl alcohol (10 mL), and then dried under vacuum at 45° C. to obtain the title compound.

Yield: 0.46% w/w

Chromatographic purity: 98.1%

1. A process for the preparation of a compound of Formula IV 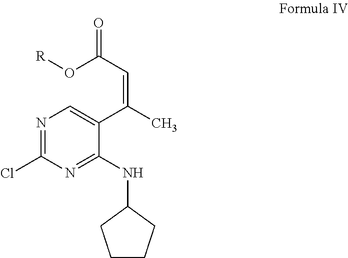
wherein R is trimethylsilyl, dimethylsilyl, or tert-butyldimethylsilyl
comprising reacting a crotonic acid derivative of Formula V 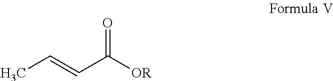
wherein R is trimethylsilyl, dimethylsilyl, or tert-butyldimethylsilyl
with a compound of Formula III 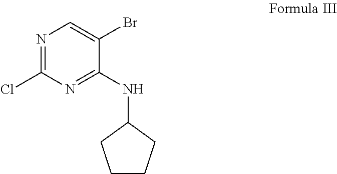 [SMILES: Clc1ncc(Br)c(NC2CCCC2)n1]
in the presence of a palladium catalyst, a base, and optionally a ligand to give a compound of Formula IV.

2. The process according to claim 1, wherein the compound of Formula IV is further converted to palbociclib of Formula I.

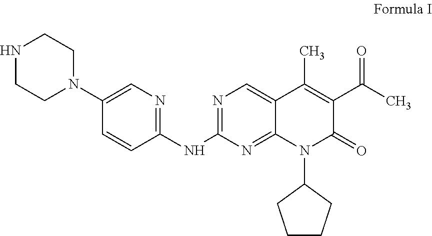 [SMILES: CC(=O)c1c(C)c2cnc(Nc3ccc(N4CCNCC4)cn3)nc2n(C2CCCC2)c1=O]

3. The process according to claim 1, further comprising intramolecular cyclization of the compound of Formula IV to give a compound of Formula II.

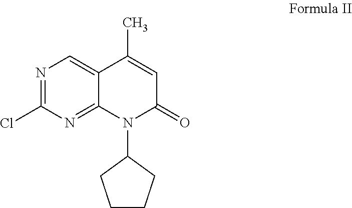 [SMILES: Cc1cc(=O)n(C2CCCC2)c2nc(Cl)ncc12]

4. The process according to claim 3, wherein the compound of Formula II is further converted to palbociclib of Formula I.

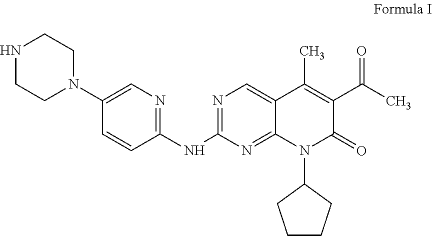 [SMILES: CC(=O)c1c(C)c2cnc(Nc3ccc(N4CCNCC4)cn3)nc2n(C2CCCC2)c1=O]

5. The process according to claim 1, wherein the compound of Formula III is reacted with the compound of Formula V to give the compound of Formula IV in a solvent.

6. The process according to claim 1, wherein the palladium catalyst is selected from the group consisting of tetrakis(triphenylphosphine) palladium (0), palladium acetate, palladium chloride, and trans-dichlorobis(acetonitrile) palladium (II).

7. The process according to claim 1, wherein the base is an organic base or an inorganic base.

8. The process according to claim 7, wherein the organic base is selected from the group consisting of triethylamine, diisopropylethylamine, and tributylamine, and the inorganic base is selected from the group consisting of potassium carbonate, sodium carbonate, and lithium carbonate.

9. The process according to claim 1, wherein the ligand is selected from the group consisting of tri-o-tolylphosphine, triphenylphosphine, and tri-t-butylphosphine.

10. The process according to claim 5, wherein the solvent is selected from the group consisting of ethers, halogenated hydrocarbons, alcohols, and esters.

11. The process according to claim 3, wherein the intramolecular cyclization of the compound of Formula IV to give the compound of Formula II is carried out in the presence of an acid anhydride or an acid chloride.

12. The process according to claim 11, wherein the acid anhydride is selected from the group consisting of acetic anhydride, propionic anhydride, butyric anhydride, trifluoroacetic anhydride, and trifluoromethanesulfonic anhydride, and the acid chloride is selected from the group consisting of acetyl chloride and ethanoyl chloride.
